(19) United States
(12) Patent Application Publication
(10) Pub. No.: US 2008/0269582 A1
(43) Pub. Date: Oct. 30, 2008

(54) Methods and devices for surgical drains with sensors

(76) Inventors: Hebah Noshy Mansour, La Mirada, CA (US); Mona Bahgat Zaki Mikhail, La Mirada, CA (US)

Correspondence Address: HEBAH NOSHY MANSOUR, 14759 HARDAWAY DR., LA MIRADA, CA, 90638, US

(21) Appl. No.: 12/150,580

(22) Filed: Apr. 28, 2008

Related U.S. Application Data
(60) Provisional application No. 60/926,641, filed on Apr. 28, 2007.

(51) Int. Cl. A61B 5/05; A61M 27/00

(52) U.S. Cl. 600/357; 604/541; 600/345; 600/361

(57) ABSTRACT

The present invention is directed to a system and method for postoperative monitoring of the condition of a tissue or organ utilizing sensors that may be embedded in various types of surgical drains. The system is comprised of a probe and a monitoring unit. The probe may include a surgical drain with fluid draining channels housing one or more sensors to measure various parameters of the adjacent tissue. The monitoring unit which controls the sensors of the probe may include a processor to process, record and display the measured parameters. This system may be valuable for monitoring transplanted organs and tissue grafts during the critical postoperative period when most of the clinical complications, such as vascular thrombosis, may occur.

### CROSS-REFERENCE TO RELATED APPLICATION(S)

This application claims priority to U.S. Provisional Patent Application 60/926,641 filed Apr. 28, 2007, entitled Methods and Devices for Surgical Drains with Sensors, the entire contents of which are incorporated herein by reference.

### FIELD OF THE INVENTION

The present invention is directed to devices and methods of using a surgical drain to monitor internal tissue condition, and more particularly to a surgical drain having at least one sensor for monitoring the condition of a tissue proximate to the surgical drain.

### BACKGROUND OF THE INVENTION

Vascular complications may occur after organ transplantation which can compromise the survival of the organ and, in some cases, the patient. Surgical resection of some organs such as the liver may introduce vascular complications to the remaining portion of the organ depending on the type and extent of the resection. This makes it important to monitor the surgically affected organs during the postsurgical period for the early detection of complications which may enable organ-saving intervention before the occurrence of irreversible tissue damage or total organ loss.

For example, monitoring of hepatic oxygenation is essential after liver transplantation and resection. Currently, the measurement of the liver enzymes and clotting factors via blood analysis is the only reliable way to monitor liver dysfunction. Changes in these laboratory values can be detected only after significant liver damage has already occurred and hence intervention usually takes place retrospectively. Also, these tests have no dynamic value since they indicate the liver condition only at the time when the blood sample is withdrawn.

Current organ monitoring technology offers probes that may require stitching or gluing to the tissue and therefore may not be easy to apply or remove especially if used inside the body. Probe stitching to the surface of an organ may also disturb the local microvasculature, cause subcapsular hematoma, and interfere with the measurement of the probe. Following are some examples of commercially available organ and tissue monitoring technologies.

Thermodilution organ monitoring technology uses a catheter-like probe that is inserted into the organ to measure its perfusion using thermodilution. The tip of the catheter-like probe includes a thermistor that is heated to remain slightly above the tissue temperature. The local perfusion is estimated from the power used in heating the thermistor, which generally depends on the ability of the tissue to dissipate heat by both thermal conduction within the tissue and by thermal convection due to tissue blood flow. This organ-invasive probe may cause bleeding, subcapsular hematoma, and may require extra care during insertion to avoid the puncture of underlying vessels.

Doppler ultrasound graft monitoring technology uses a suturable cuff probe that is fitted around the vessels supplying the tissue to assess its blood flow using Doppler ultrasound. Post-monitoring, the cuff probe may be difficult to remove and may left permanently around the vessel.

Optical tissue monitoring technology uses button-like probes are stitched to the tissue to measure its oxygen saturation using reflectance spectroscopy (e.g. Stitching can complicate probe application and removal. Also, stitching may disturb the local microcirculation and introduces measurement errors.

Laser Doppler Flowmetry tissue monitoring technology uses button-like probes are stitched to the tissue to measure its blood perfusion using laser Doppler flowmetry. Again, stitching can complicate probe application and removal and disturb the local microcirculation thereby introducing measurement errors.

Surgical drains (or surgical wound drains, used interchangeably herein) are routinely implanted during many surgical procedures to drain the wound fluids out of the body during the postoperative period. Some well-known examples of the surgical drains are and the Blake drains (e.g. U.S. Pat. Des. 288,962, U.S. Pat. No. 4,398,910, and U.S. Pat. No. 4,465,481). Surgical drains are generally used with a vacuum source to enhance the draining of the wound fluids out of the body.

The current invention relates to probes for monitoring the condition of tissues and organs. This may be useful for monitoring transplanted tissues and organs during the critical postoperative period when most of the clinical complications, such as vascular thrombosis, may occur.

The invention discloses a surgical drain with sensors (or drain probe, used interchangeably herein) for the monitoring of organs and tissues with possible applications in plastic and reconstructive surgery, general surgery, resection surgery and transplant surgery.

The disclosed drain probe may benefit from the suction applied to the surgical drains to facilitate the draining of the wound fluid. This suction creates vacuum that may bring the drain probe and the adjacent tissue together thereby holding the drain probe in position and maintaining good contact between its probe's sensors and the tissue. Another benefit of this suction may be the continuous clearing of the local wound fluid that may otherwise insulate the sensors of the drain probe from the adjacent tissue and therefore impede their measurement.

Depending on the application, the drain probe may include sensors to measure the oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure.

The drain probe connects to a monitoring unit that drives the sensors, processes the sensor data, and displays the measured parameters.

The present invention describes several embodiments of the drain probe, which may be suitable for monitoring local tissue and organs after various surgical procedures.

### SUMMARY OF THE INVENTION

The present invention discloses a system and method for postoperative monitoring of the condition of a tissue (or organs, used interchangeably herein) utilizing sensors that may be embedded in various types of surgical drains. The system is comprised of a probe and a monitoring unit.

The probe may include sensors to measure parameters of the tissue and a surgical drain with elongated channels (or grooves, used interchangeably herein) that allow the collection and drainage of the wound fluids.

Depending on the monitoring application, the drain probe may incorporate sensors to measure the oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure. For example, percent oxygen saturation may be the preferred parameter for monitoring transplanted organs and tissue grafts which may be susceptible to thrombosis in their newly connected vessels.

The monitoring unit which controls the sensors of the probe may include a processor to process, record and display the sensor data.

The probe may be implanted in the body next to the tissue to be monitored, and the probe anchored at the desired position by the vacuum-induced compression of the surrounding tissues created by the suction action of the surgical drain.

These, as well as other objects, features and benefits will now become clear from a review of the following detailed description of illustrative embodiments and the accompanying drawings.

### BRIEF DESCRIPTION OF THE DRAWINGS

FIG. 1 is a schematic diagram of one embodiment of a surgical drain having at least one sensor and a monitoring unit in accordance with the present invention.

FIG. 2 is a schematic diagram of one embodiment of a surgical drain having at least one sensor and a monitoring unit in accordance with the present invention.

FIG. 3 is a schematic diagram of one embodiment of a surgical drain in use having at least one sensor and a monitoring unit in accordance with the present invention.

FIG. 4 is a schematic diagram of one embodiment of a surgical drain having at least one sensor and a monitoring unit in accordance with the present invention.

### DETAILED DESCRIPTION OF THE INVENTION

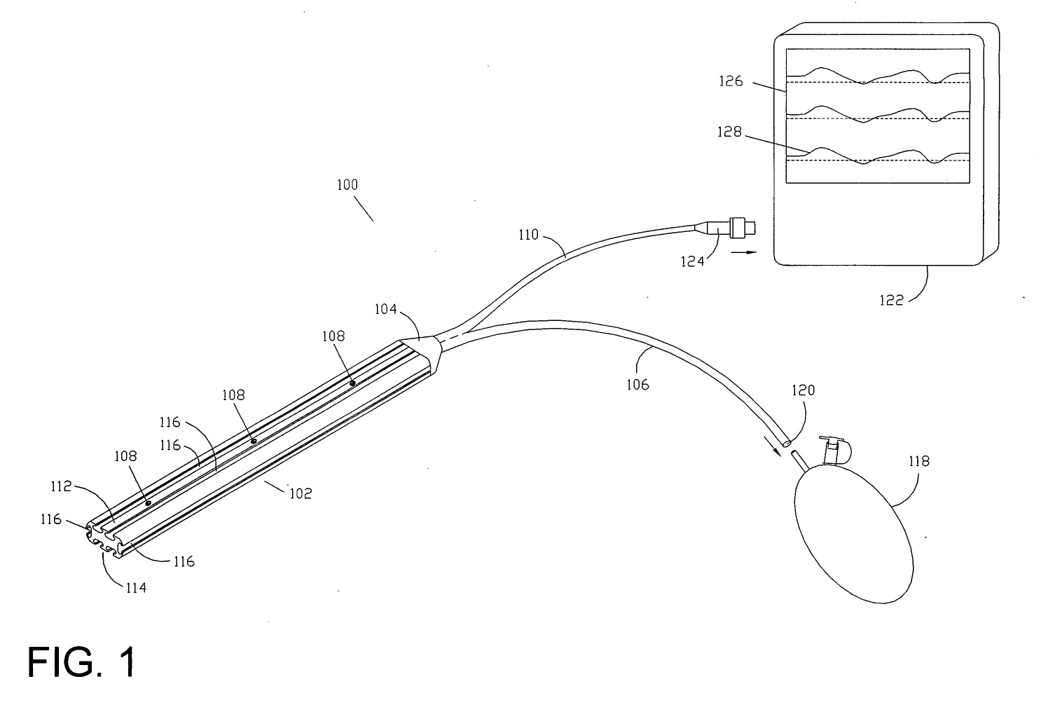

One embodiment of the drain probe 100 is shown in FIG. 1. The drain probe 100 may be comprised of a drain body 102, a fluid collection funnel 104, a fluid draining tube 106, sensors 108, and data cable 110. The drain body 102, collection funnel 104, and draining tube 106 may be made of flexible material such as medical-grade silicone or other elastomeres. The drain body may be made of a radiopaque material such as barium-loaded medical grade silicone for easier detection using radiographic techniques.

The drain body 102 may be preferably flat with a cross-section that is approximately rectangular in shape with a first surface 112 and a second surface 114. Alternatively, the cross-section of the drain body 102 may be square, elliptical, semi-circular, semi-elliptical, or trapezoidal in shape. A drain body 102 with a semi-elliptical or semi-circular cross-section may be advantageous in increasing the contact pressure between the sensors 108 and the adjacent tissue whereas the sensors 108 may be located on the line-apex of the drain body 102.

The channels 116 may be grooves or slits in the drain body 102 and may have different cross-sectional shapes including square, rectangular, semi-circular, semi-elliptical, triangular, semi-triangular, trapezoidal, C-shaped, V-shaped, U-shaped, and L-shaped. The channels 116 may run along the entire length (or part of the length) of the drain body 102 and are in fluid communication (or hydraulic continuity) with the collection funnel 104 and the draining tube 106. The channels 116 may collect the wound fluid from the tissue areas local to the drain probe 100 and stream the wound fluid into the collection funnel 104.

The collection funnel 104 gathers the wound fluid streamed through the channels 116 and funnels the wound fluid into the draining tube 106. The draining tube 106 transports the wound fluid out of the body into a collection reservoir or bulb 118. External suction (or negative pressure) may be applied to the in-vitro end 120 (i.e. out of the body end) of the fluid draining tube 106 to facilitate the draining of the wound fluid out of the body.

The sensors 108 may be located on the first surface 112 of the drain body 102 to measure one or more parameters of the tissues adjacent to the first surface 112. The sensed parameters of the tissues may include: oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure.

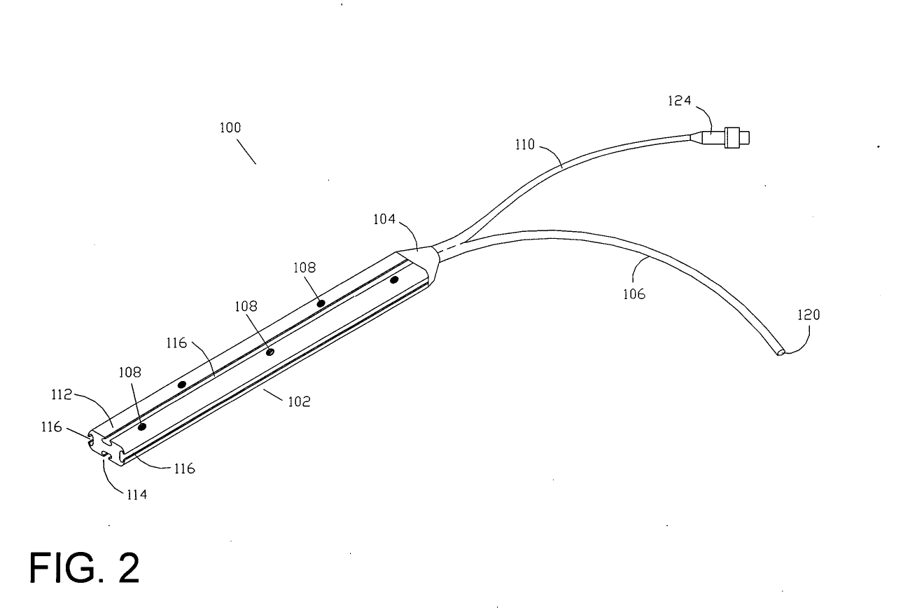

The sensors 108 may be located in the center isle between two of the channels 116 in the first surface 112 as shown FIG. 1, or on both the sides of a single channel 116 in the first surface 112 as shown FIG. 2.

The sensors 108 may be located on both the first surface 112 and the second surface 114 to monitor a first parameter of a first tissue that is adjacent to the first surface 112 and a second parameter of the second tissue that is adjacent to the second surface 114, respectively. The first and second parameters may be similar or different.

The comparison of the same parameter measured from different first and second tissues may provide useful diagnostic information. For example, sensors on the first surface 112 may be measuring the percent oxygen saturation of a native tissue with intact blood vessels while other sensors on the second surface 114 may be measuring the percent oxygen saturation of a transplanted tissue with newly connected blood vessels. A mutual decrease in the percent oxygen saturation measured by the sensors of the first surface and the sensors of the second surface may indicate that this decrease in percent oxygen saturation is due to a global (i.e. whole body) decrease in the percent oxygen saturation or blood perfusion. However, a unilateral decrease in the percent oxygen saturation measured by the sensors of the second surface from the transplanted tissue may indicate an occlusion or thrombosis in the newly connected blood vessels supplying the transplanted tissue.

The sensors 108 may be also placed in the channels 116 and/or the collection funnel 104 to monitor the composition of the wound fluid being drained. Changes in the composition of the wound fluid over time (i.e. comparing change over time) may provide useful diagnostic information.

For example, a sensor 108 monitoring the change in hemoglobin concentration in the wound fluid may allow the detection of internal bleeding or wound healing complications. Normally, the concentration of hemoglobin in the wound fluid is expected to decrease with time after surgery. However, an increase in concentration the hemoglobin in the wound fluid may be indicative internal bleeding or leaking vessels.

The optical absorption characteristics of the hemoglobin in the wound fluid may be indicative whether the source of the internal bleeding is arterial or venous. Furthermore, the rate of change in the concentration of hemoglobin may be indicative of the severity of the internal bleeding. A high rate of increase in hemoglobin concentration in the wound fluid may indicate severe internal bleeding and vise versa. The processor within the monitoring unit 122 may process the concentration and the rate of change of concentration of different substances (e.g. hemoglobin, bilirubin, amylase, intestinal content, pus, etc.) in the wound fluid to determine a condition of the surgical wound and/or the adjacent tissues (or organs).

The sensors 108 may be of the optical, electrical, electromechanical and/or electrochemical types. In addition, the sensors may be a tube that hydraulically transmits the internal pressure to the outside of the body where it may be measured using a pressure transducer.

The sensors 108 may be of the type that requires transmitting energy to the tissue and receiving the energy portion returned from the tissue. For example, a fiberoptic oximetry sensor may be composed of at least a first and second optical fibers embedded in the surgical drain for transmitting light to and from the internal tissue adjacent to the surgical drain. The first optical fiber may transmit light from a light source (for example a lamp, a laser, or a light emitting diode) to the tissue and the second optical fiber may collect the light portion returned from the tissue and transmit it back to an external photodetector (e.g. photodiode or a spectrometer). The light returned from the tissue may be processed by a processor to determine the hemoglobin content and oxygen saturation of the tissue. Furthermore, the spectral differences between the transmitted and the received light may be compared to determine the hemoglobin content and oxygen saturation of the tissue. The distal apertures of the first and second optical fibers may be isolated from the adjacent tissue by an optically transparent window in the drain wall. The optically transparent window may be made of an optically transparent medical-grade silicone.

The sensors 108 may communicate through the data cable 110 with the monitoring unit 122. The data cable 110 may be electrical and/or fiberoptic and may be covered by medical grade silicone sleeve and ends with a connector 124. The data cable 110 may be attached to the draining tube 106 for a given distance from the funnel 104 until they branch away from each other.

The monitoring unit 122 may include drivers to control and read the sensors, a processor to process the data from the sensor, and a display 126 to display the processed data from the sensor as a graphical trace 128 and/or alphanumeric numbers.

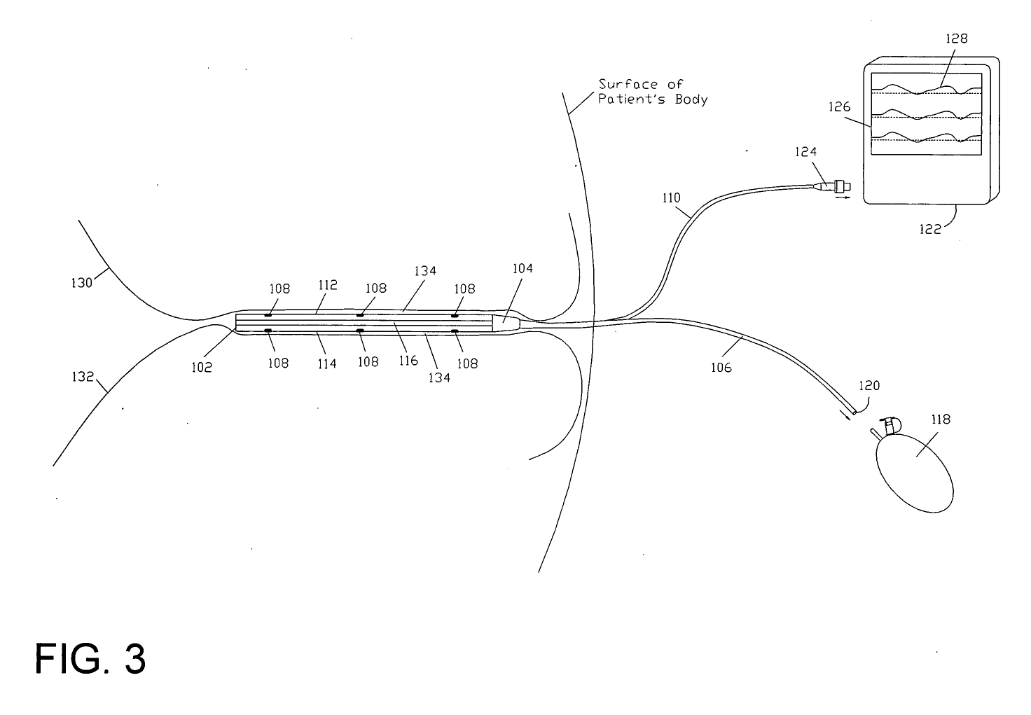

An example of the application of the drain probe 100 is shown in FIG. 3. At the end of the surgical procedure, the drain probe 100 is placed in the surgical wound. The drain probe 100 is positioned between a first tissue 130 and a second tissue 132 within the surgical wound whereas the first tissue 130 and second tissue 132 may be parts of the same tissue or parts of different tissues. The surgical wound is closed as in routine surgical practice and suction may be applied to the end 120 of the fluid draining tube 106 to remove the wound fluid.

The end 120 may be connected to a squeezable/self-expandable fluid collection bulb or reservoir 118. The applied suction may bring together the first tissue 130 and the second tissue 132 to hold in-between the probe 100 and maintain its position. In addition, the applied suction may also clear the wound fluid from the interface space 134 between the tissues 130 and 132 and the drain body 102 which allows better coupling between the sensors 108 and the adjacent tissues 130 and 132.

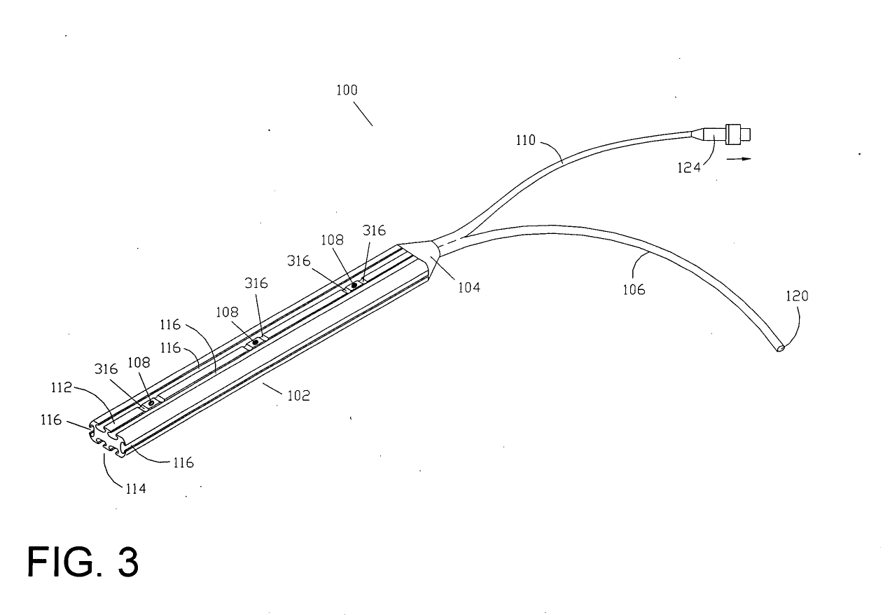

FIG. 4 shows an alternative embodiment of the drain probe 100 where minor channels 316 are added to the drain body 102 just around the locations of the sensors 108 to enhance the removal of the wound fluid from the location of the sensors 108. The minor channels 316 are in fluid communication (or hydraulic continuity) with the major channels 116 such that any wound fluid collected by the minor channels 316 is directly streamed into the channels 116.

Furthermore, the locations of the sensors 118 may be slightly elevated above the level of the first surface 112 to improve the contact pressure between the sensors 108 and the adjacent tissue.

Although the above detailed description describes and illustrates various preferred embodiments, the invention is not so limited. Many modifications and variations will now occur to persons skilled in the art. As such, the preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

What is claimed is:

1. A surgical drain system for draining wound fluids and sensing a parameter of a tissue in a patient's body comprising:
a) a surgical drain configured to be implanted in a patient's body, to rest against at least one tissue in the patient's body, to house at least one sensor, and to drain wound fluids from the vicinity of the tissue, comprising:
i. a drain body configured to rest against the tissue within the patient's body;
ii. a first surface located on an outer side of the drain body;
iii. one or more draining grooves along substantially the length of the drain body;
iv. a first sensor integrated with the first surface, configured to sense a parameter of the tissue proximate to the first surface; and
b) a tube in fluid communication with the surgical drain configured to transport the drained wound fluids out of the body.

2. The system of claim 1, wherein the parameter sensed is selected from the group comprising: oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure.

3. The system of claim 1, wherein the first sensor detects the level of oxygenation of the tissue.

4. The system of claim 1, wherein the first sensor detects the hemoglobin content in the tissue.

5. The system of claim 1, wherein the sensor includes at least one optical fiber.

6. The surgical drain of claim 1, further including a transmitting element configured to deliver energy to the tissue proximate the first surface.

7. The system of claim 1, wherein the first sensor is configured to sense the parameter by sensing energy that is returned from the tissue after having been transmitted to the tissue.

8. The system of claim 1, further comprising a second sensor integrated with the first surface, configured to detect a parameter of the tissue that is different from the parameter sensed by the first sensor.

9. The system of claim 1, wherein the first sensor is embedded within the surgical drain behind material that is optically transparent.

10. The system of claim 1, further including display configured to depict data corresponding to the parameter sensed by the first sensor.

11. The system of claim 1, wherein the surgical drain further includes a second surface located on an outer side of the drain body different from the first surface and a second sensor integrated with the second surface, configured to sense the same parameter of a tissue proximate to the second surface which is different from the tissue sensed by the first sensor.

12. The system of claim 11, further including a processor in communication with the first and the second sensors, and configured to compare the difference between the parameter sensed by the first and the second sensors.

13. A surgical drain system for draining wound fluids and sensing a parameter of a tissue in a patient's body comprising:
a) a surgical drain configured to be implanted in a patient's body, to rest against at least one tissue in the patient's body, to house at least one sensor, and to drain wound fluids from the vicinity of the tissue, comprising:
i. a drain body configured to rest against the tissue within the patient's body;
ii. a first surface located on an outer side of the drain body and a second surface located on an outer side of the drain body different from the first surface;
iii. one or more draining grooves along substantially the length of the drain body;
iv. a first sensor integrated with the first surface, configured to sense a parameter of a first tissue proximate to the first surface; and
v. a second sensor integrated with the second surface, configured to sense the same parameter of a second tissue proximate to the second surface;
b) a tube in fluid communication with the surgical drain configured to transport the drained wound fluids out of the body; and
c) a processor in communication with the first and the second sensors configured to compare the difference between the parameter sensed by the first and the second sensors.

14. The system of claim 13, wherein the parameter sensed is selected from the group comprising: oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure.

15. The system of claim 13, wherein the first sensor is configured to sense the parameter by sensing energy that is returned from the tissue after having been transmitted to the tissue.

16. The system of claim 13, wherein the first and the second sensing systems are configured to sense the parameter by sensing energy that has been returned from tissue after having been transmitted into the tissue.

17. The system of claim 13 further including a display configured to depict data corresponding to the parameter sensed by the first and the second sensors.

18. The system of claim 13, further including a display configured to depict data corresponding to a difference between the parameter sensed by the first and second sensors.

19. A method of utilizing a surgical drain to monitor a condition of a tissue in a patient's body, comprising:
a) implanting the surgical drain in the patient's body, wherein the surgical drain comprising:
i. a drain body configured to rest against the tissue within the patient's body;
ii. a first surface located on an outer side of the drain body;
iii. one or more draining grooves along substantially the length of the drain body configured to drain wound fluids;
iv. one or more sensors integrated with the first surface, configured to sense one or more parameter of the tissue proximate to the first surface; and
v. a tube in fluid communication with the drain body configured to transport the drained wound fluids out of the body.
b) applying external suction to the surgical drain for draining the wound fluids from the vicinity of the tissue;
c) sensing one or more parameter of the tissue proximate to the first surface; and
d) processing and displaying the one or more parameter of the tissue.

20. The method of claim 19, wherein the sensed one or more parameter is selected from the group comprising: oxygen partial pressure, percent oxygen saturation, hemoglobin concentration, blood perfusion, pH, NADH concentration, humidity, biochemical composition, bilirubin concentration, amylase concentration, pus, intestinal content, drug concentration, temperature and pressure.
